(19) United States
(12) Patent Application Publication
(10) Pub. No.: US 2017/0071516 A1
(43) Pub. Date: Mar. 16, 2017

(54) MOBILE OPTICAL DEVICE AND METHODS FOR MONITORING MICROVASCULAR HEMODYNAMICS

(71) Applicant: Samsung Electronics Co., Ltd., Suwon-si (KR)

(72) Inventors: Yusuf A. Bhagat, Mountain View, CA (US); Sean D. Lai, Mountain View, CA (US); Insoo Kim, Mountain View, CA (US)

(21) Appl. No.: 14/988,619

(22) Filed: Jan. 5, 2016

Related U.S. Application Data
(60) Provisional application No. 62/218,915, filed on Sep. 15, 2015.

(51) Int. Cl. A61B 5/1455; A61B 5/026; A61B 5/00; A61B 5/021

(52) U.S. Cl. CPC A61B 5/14551; A61B 5/02141; A61B 5/0261; A61B 5/743; A61B 5/6898

(57) ABSTRACT

A method implemented using a device to measure hemodynamic parameters is provided. The method includes transmitting, by the client device, a message to the server. The method includes capturing, by a camera, a first image of a plurality of images of a target region while two light emitting diode (LED) sensors emit light, via a collimated lens, on the target region. The method also includes capturing, by the camera, a second image of the plurality of images of the target region while the two LED sensors emit light, via the collimated lens, on the target region. The second image is captured a predetermined time after the first image is captured. The method further includes determining one or more hemodynamic parameters based on a difference between the first captured image and the second captured image.

### CROSS-REFERENCE TO RELATED APPLICATION(S) AND CLAIM OF PRIORITY

The present application claims priority to U.S. Provisional Patent Application Ser. No. 62/218,915, filed Sep. 15, 2015, entitled “MOBILE OPTICAL DEVICE AND METHODS FOR MONITORING MICROVASCULAR HEMODYNAMICS”. The content of the above-identified patent document is incorporated herein by reference in its entirety.

### TECHNICAL FIELD

The present application relates generally to monitoring bodily parameters and, more specifically, to a monitoring bodily parameters using a mobile electronic device.

### BACKGROUND

Smartphones and accompanying wearable devices include self-monitoring and quantification features to obtain physiological parameters. These devices use noninvasive measurement means to measure heart rate (HR), heart rate variability (HRV), and oxygen saturation in the blood (Sp0₂). Improvements to such smartphones and accompanying devices can be implemented to measure additional bodily parameters.

### SUMMARY

A device to measure hemodynamic parameters is provided. The device includes a pair of light emitting diode (LED) sensors configured to emit light. The two LED sensors are covered with a collimated lens. The device further includes a camera. The device further includes a processor. The processor is configured to control the camera to capture a first image of a target region while the LED sensors emit light on the target region. The processor is also configured to control the camera to capture a second image of the target region while the LED sensors emit light on the target region. The second image is captured a predetermined time after the first image is captured. The processor is further configured to determine one or more hemodynamic parameters based on a difference between the first captured image and the second captured image.

A device to measure hemodynamic parameters is provided. The device includes a pair of light emitting diode (LED) sensors configured to emit light. The LED sensors are covered with a collimated lens. The device further includes a camera. The device further includes a processor. The processor is configured to control the camera to capture a first image of a target region while the LED sensors emit light on the target region. The processor is also configured to control the camera to capture a second image of the target region while the LED sensors emit light on the target region. The second image is captured a predetermined time after the first image is captured. The processor is further configured to receive a selection to perform at least one of particle image velocimetry (PIV) imaging or photoplethysmography (PPG) imaging. In addition, the processor is configured to determine one or more hemodynamic parameters based on (1) a difference between the first captured image and the second captured image and (2) the received selection.

A method implemented using a device to measure hemodynamic parameters is provided. The method includes transmitting, by the client device, a message to the server. The method includes capturing, by a camera, a first image of a target region while a pair of light emitting diode (LED) sensors emit light, via a collimated lens, on the target region. The camera can be a high resolution camera. The method also includes capturing, by the camera, a second image of the target region while the two LED sensors emit light, via the collimated lens, on the target region. The second image is captured a predetermined time after the first image is captured. The method further includes determining one or more hemodynamic parameters based on a difference between the first captured image and the second captured image.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely. The phrase “at least one of,” when used with a list of items, means that different combinations of one or more of the listed items may be used, and only one item in the list may be needed. For example, “at least one of: A, B, and C” includes any of the following combinations: A, B, C, A and B, A and C, B and C, and A and B and C.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 illustrates an example communication system according to this disclosure;

FIGS. 2 and 3 illustrate example devices in a communication system according to this disclosure;

FIG. 4 illustrates an example cross-sectional diagram of anatomy of a human epidermal layer according to this disclosure;

FIGS. 5A and 5B illustrate an example electronic device including a combined particle image velocimetry (PIV) and photoplethysmography (PPG) imaging system according to this disclosure;

FIG. 6 illustrates an example system block diagram of an example electronic device according to this disclosure;

FIG. 7 illustrates an example microscopic PIV system according to this disclosure;

FIG. 8 illustrates an example method implemented using a microscopic PIV system according to this disclosure;

FIG. 9 illustrates an example method of image sensing using a microscopic PIV system according to this disclosure;

FIG. 10 illustrates an example of a PPG imaging system according to this disclosure;

FIG. 11 illustrates an example method to compute a final PPG imaging color maps for displaying the AC amplitude of the PPG signal on a pixel-by-pixel basis according to this disclosure;

FIG. 12 illustrates an example method for demonstrating the operation of the image sensors when combining the PIV and PPG imaging systems using an electronic device according to this disclosure;

FIGS. 13A, 13B and 13C illustrate an example process to measure microvascular hemodynamic parameters according to this disclosure; and

FIG. 14 illustrates an example process to measure microvascular hemodynamic parameters according to this disclosure.

### DETAILED DESCRIPTION

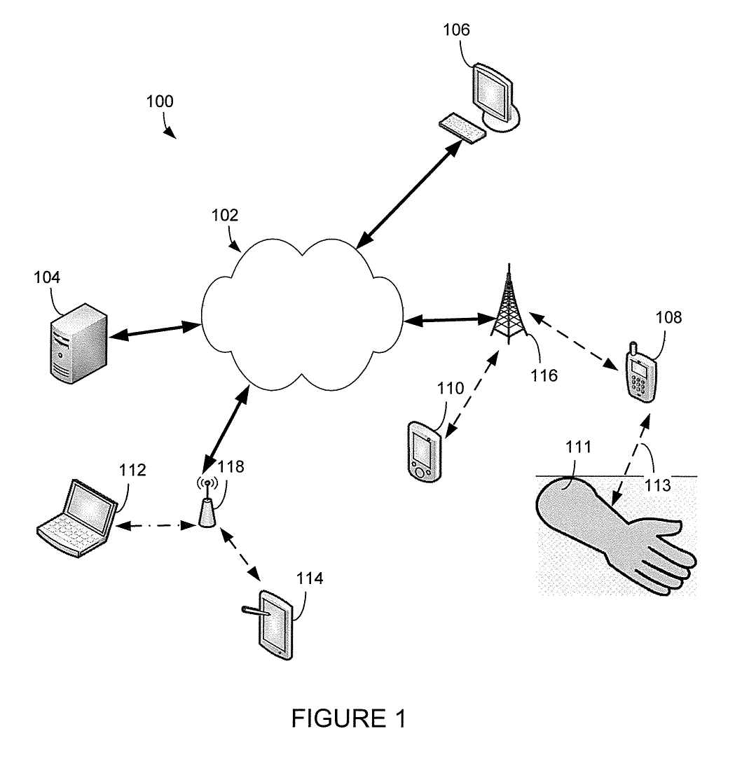

FIGS. 1 through 14, discussed below, and the various embodiments used to describe the principles of the present invention in this patent document are by way of illustration only and should not be construed in any way to limit the scope of the disclosure. Those skilled in the art will understand that the principles of this disclosure may be implemented in any suitably arranged device or system.

The proliferation of smartphones and accompanying wearable devices has made self-monitoring and quantification of physiological parameters more accessible and affordable. As discussed herein, such devices can noninvasively measure an individual's heart rate (HR) on the basis of photoplethysmography (PPG) sensors which utilize light emitting diodes (LED) by illuminating the skin and measuring changes in light absorption via a photodiode. Additionally PPG sensors can also be used for measuring heart rate variability (HRV) and provide pulse oximetry, which yields oxygen saturation levels (SpO₂). The extent of parameters reflecting an individual's circulatory condition on the basis of PPG sensors is quite narrow and limited to these three aforementioned metrics (HR, HRV and SpO₂). Hemodynamic parameters reflecting the circulatory condition of an individual such as blood velocity, flow, cardiac output, turbulence, wall tension, vessel capacitance and ultimately, blood pressure provide more insights into an individual's cardiovascular fitness or lack thereof.

As discussed herein electronic devices (such as smartphones) can measure cardiovascular parameters in addition to HR, HRV and SpO₂ including velocity, flow, and blood pressure. Pulsed LEDs juxtaposed to a camera on the back of a smartphone can be used to focus a collimated light beam on a small field of view of an anatomical structure (such as a hand or a finger) for capturing a change in blood flow that can then be recorded by the camera. Filtering, reconstruction, and cross-correlation techniques can then provide vectograms showing a vector field map within the field of view (FOV) that can also be used to output a velocity of the blood in that region of interest. Furthermore, the same electronic device can be used to measure variations in heart rate by calculating the alternating current amplitude and pulse rate within the same FOV to provide a PPG image map of heart rate and also SpO₂. The parameter ensemble can then be used to gather estimates of individual blood pressure.

Also, as discussed herein, an electronic device can include LEDs with pulsing properties next to a high definition (1080p, 60 frames per second (fps)) camera on a rear surface of the electronic device that can produce a collimated beam of light that can be aimed at any superficial anatomical region for imaging and measuring multiple hemodynamic parameters including heart rate, heart rate variability, SpO₂, blood flow velocity, and the like. Such parameters not only provide insights into distinct cardiovascular system measurements, but can also be used collectively to estimate blood pressure without the encumbrance of cuff-based devices. Using an electronic device as discussed herein vectograms or vector overlays of blood flow within an anatomical region can be outputted as well as imaging of the heart rate variability and oxygen saturation in that same anatomical region can be outputted.

The interaction of light with biological tissue is complex and includes optical processes such as scattering, absorption, reflection, transmission, and fluorescence. Photoplethysmography (PPG) is a noninvasive optical measurement method operating at a red or near infrared wavelength used for detecting blood volume changes in the microvascular bed of tissue. PPG requires a few opto-electronic components in the form of a light source for illuminating the tissue (skin) and a photodetector to measure the small variations in light intensity resulting from changes in perfusion in the measurement volume. The peripheral pulse as seen in a PPG waveform is synchronized to each heartbeat. The pulsatile component of the PPG waveform is referred to as the alternating current (AC) component with frequency at ˜1 Hz and is super-imposed on to a large quasi direct current (DC) component associated with tissues and the average blood volume. Factors influencing the DC component are respiration, vasomotor activity, and thermoregulation. Appropriate filtering and amplification techniques permit extraction of both, the AC and DC components for pulse wave analysis. Pulses recorded via PPG sensors are linearly related to perfusion with a higher blood volume attenuating the light source to a greater extent.

Light emitting diodes (LED) which comprise the light source of PPG sensors have a narrow bandwidth (˜50 nm) and convert electrical energy into light energy. Advantages of LEDs are compactness, long operating life (10⁵ hours) over a wide temperature range, robustness and reliability. The average intensity of LEDs is low enough to prevent local tissue heating and risks of non-ionizing radiation. Photodetectors used with LEDs are selected with similar spectral characteristics and convert light energy into an electrical current. They too are compact, low-cost, sensitive, and have fast response times. PPG sensors can be held securely against the skin to minimize probe-tissue motion artifacts, which can cause variations in the blood volume signal measured. Excessively tight coupling between the probe and tissue can impede circulation and dampen the pulse wave response. A PPG system incorporating LEDs and a camera for distance imaging of beat-to-beat pressure may provide a robust device.

Particle image velocimetry (PIV) is a fluid dynamics-based technique that measures the displacement of fluid over a finite time interval. The position of the fluid is imaged through light scattered by liquid or solid particles illuminated by a laser (such as Neodymium-doped yttrium aluminium garnet (Nd:YAG)) light sheet. For some PIV applications, such particles are not naturally present in the flow of interest and therefore need to be seeded with tracer particles that move with the local flow velocity. Pulsed Nd:YAG laser beams (λ, 532 nm; duration, 5-10 nanoseconds; energy, ˜400 mJ/pulse) are superimposed so that two laser sheets illuminate the same area or field of view. A charge coupled device (CCD) camera sensor is used for digital image recording where photons are converted to an electric charge based on the photoelectric effect. The light scattered by the particles is recorded on two separate frames of the CCD camera. A cross-correlation function based on Fast Fourier transform (FFT) algorithms is used to estimate the local displacement vector of particle images between two illuminations for each area or “interrogation window” of the digital PIV recording. Based on the time interval between the two laser pulses and the image magnification from the camera calibration, a projection of the local flow velocity vector on to the plane of light sheet can then be deduced.

PIV systems that are used for industrial flow applications can have laser diode modules that provide sufficient power and high geometrical beam quality for producing a very thin light sheet for each sequential interrogation window. Furthermore, several cameras can also be used to not only generate vector field projections of flowing liquids in multiple dimensions, but to also perform tomographic PIV scanning of a flowing medium. Laser-based PIV systems can have a higher cost relative to LEDs, can have an unstable pulse-to-pulse light output (such as in terms of intensity and spatial distribution), and can have uncollimated light emission and speckle artifacts. LEDs for volume illumination of a plane can be used for sound PIV systems instead.

FIG. 1 illustrates an example computing system 100 according to this disclosure. The embodiment of the computing system 100 shown in FIG. 1 is for illustration only. Other embodiments of the computing system 100 could be used without departing from the scope of this disclosure.

As shown in FIG. 1, the system 100 includes a network 102, which facilitates communication between various components in the system 100. For example, the network 102 may communicate Internet Protocol (IP) packets, frame relay frames, Asynchronous Transfer Mode (ATM) cells, or other information between network addresses. The network 102 may include one or more local area networks (LANs), metropolitan area networks (MANs), wide area networks (WANs), all or a portion of a global network such as the Internet, or any other communication system or systems at one or more locations.

The network 102 facilitates communications between at least one server 104 and various client devices 106, 108, 110, 112, or 114. Each server 104 includes any suitable computing or processing device that can provide computing services for one or more client devices. Each server 104 could, for example, include one or more processing devices, one or more memories storing instructions and data, and one or more network interfaces facilitating communication over the network 102.

Each client device 106, 108, 110, 112, or 114 represents any suitable computing or processing device that interacts with at least one server or other computing device(s) over the network 102. In this example, the client devices 106, 108, 110, 112, or 114 include a desktop computer 106, a mobile telephone or smartphone 108, a personal digital assistant (PDA) 110, a laptop computer 112, and a tablet computer 114. However, any other or additional client devices could be used in the computing system 100.

In this example, some client devices 108, 110, 112, and 114 communicate indirectly with the network 102. For example, the client devices 108-110 communicate via one or more base stations 116, such as cellular base stations or eNodeBs. Also, the client devices 112 and 114 communicate via one or more wireless access points 118, such as IEEE 802.11 wireless access points. Note that these are for illustration only and that each client device could communicate directly with the network 102 or indirectly with the network 102 via any suitable intermediate device(s) or network(s).

As described in more detail below, a client device such as client device 108 emits light 113 from one or more LEDs onto a target region 111 of a living body. The client device 108 captures an image, using a camera (such as a high-resolution camera), of the target region 111 receiving the light 113. The client device can use the data acquired by the camera to observer microvascular hemodynamic properties.

Although FIG. 1 illustrates one example of a computing system 100, various changes may be made to FIG. 1. For example, the system 100 could include any number of each component in any suitable arrangement. In general, computing and communication systems come in a wide variety of configurations, and FIG. 1 does not limit the scope of this disclosure to any particular configuration. While FIG. 1 illustrates one operational environment in which various features disclosed in this patent document can be used, these features could be used in any other suitable system.

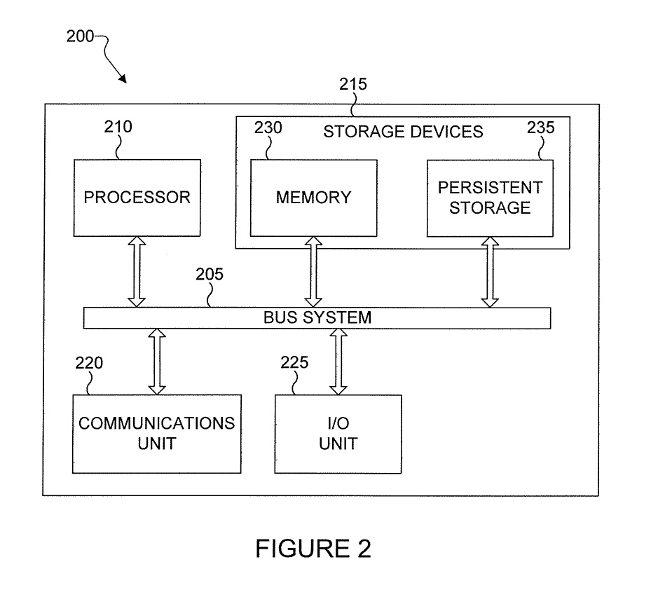

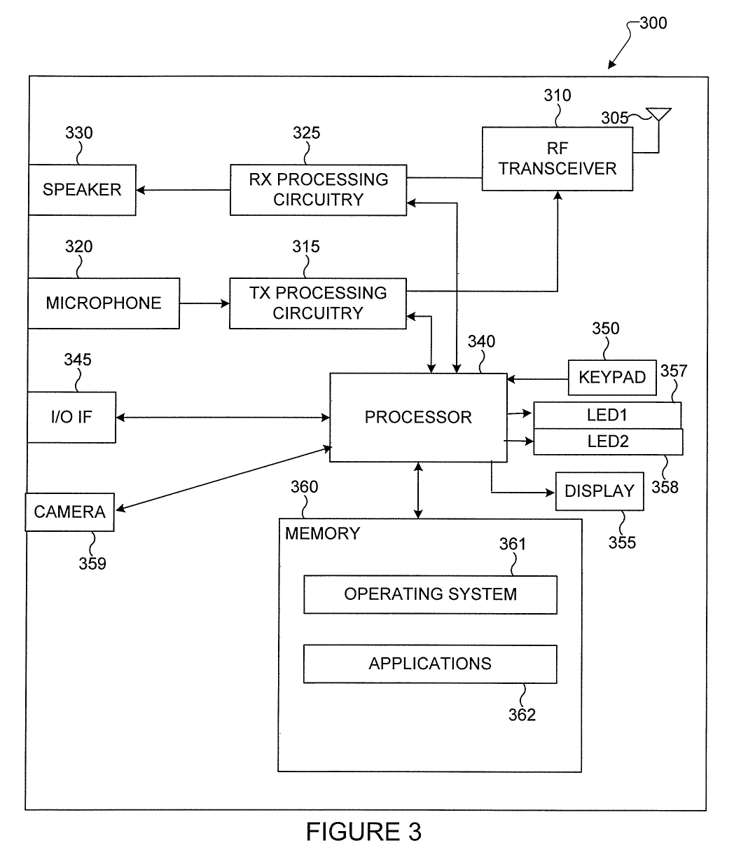

FIGS. 2 and 3 illustrate example devices in a communication system according to this disclosure. In particular, FIG. 2 illustrates an example server 200, and FIG. 3 illustrates an example client device 300. The server 200 could represent the server 104 in FIG. 1, and the client device 300 could represent one or more of the client devices 106, 108, 110, 112, or 114 in FIG. 1.

As shown in FIG. 2, the server 200 includes a bus system 205, which supports communication between at least one processor 210, at least one storage device 215, at least one communications unit 220, and at least one input/output (I/O) unit 225.

The processor 210 executes instructions that may be loaded into a memory 230. The processor 210 may include any suitable number(s) and type(s) of processors or other devices in any suitable arrangement. Example types of processors 210 include microprocessors, microcontrollers, digital signal processors, field programmable gate arrays, application specific integrated circuits, and discreet circuitry.

The memory 230 and a persistent storage 235 are examples of storage devices 215, which represent any structure(s) capable of storing and facilitating retrieval of information (such as data, program code, and/or other suitable information on a temporary or permanent basis). The memory 230 may represent a random access memory or any other suitable volatile or non-volatile storage device(s). The persistent storage 235 may contain one or more components or devices supporting longer-term storage of data, such as a ready only memory, hard drive, Flash memory, or optical disc.

The communications unit 220 supports communications with other systems or devices. For example, the communications unit 220 could include a network interface card or a wireless transceiver facilitating communications over the network 102. The communications unit 220 may support communications through any suitable physical or wireless communication link(s).

The I/O unit 225 allows for input and output of data. For example, the I/O unit 225 may provide a connection for user input through a keyboard, mouse, keypad, touchscreen, or other suitable input device. The I/O unit 225 may also send output to a display, printer, or other suitable output device.

Note that while FIG. 2 is described as representing the server 104 of FIG. 1, the same or similar structure could be used in one or more of the client devices 106-114. For example, a laptop or desktop computer could have the same or similar structure as that shown in FIG. 2.

As described in more detail below, the client device 300 and the server 200 can be used for multipath data packet transmission. For example, the client device 300 transmits a request to the server 200. The request includes an identifier that is unique to a multipath transmission session and that identifies two or more network access interfaces of the client device 300 to receive one or more data packets from the server 200 during the multipath transmission session. The client device 300 can also receive the one or more data packets from the server 200 through each of the two or more network access interfaces of the client device 300 during the multipath transmission session.

As shown in FIG. 3, the client device 300 includes an antenna 305, a radio frequency (RF) transceiver 310, transmit (TX) processing circuitry 315, a microphone 320, and receive (RX) processing circuitry 325. The client device 300 also includes a speaker 330, a processor 340, an input/output (I/O) interface (IF) 345, a keypad 350, a display 355, a light emitting diode (LED1) (at a given wavelength, λ₁) 357, an LED2 (at an alternative wavelength, λ₂) 358, a camera 359, and a memory 360. The memory 360 includes an operating system (OS) program 361 and one or more applications 362.

The RF transceiver 310 receives, from the antenna 305, an incoming RF signal transmitted by another component in a system. The RF transceiver 310 down-converts the incoming RF signal to generate an intermediate frequency or baseband signal. The intermediate frequency or baseband signal is sent to the RX processing circuitry 325, which generates a processed baseband signal by filtering, decoding, and/or digitizing the baseband or intermediate frequency signal. The RX processing circuitry 325 transmits the processed baseband signal to the speaker 330 (such as for voice data) or to the processor 340 for further processing (such as for web browsing data).

The TX processing circuitry 315 receives analog or digital voice data from the microphone 320 or other outgoing baseband data (such as web data, e-mail, or interactive video game data) from the processor 340. The TX processing circuitry 315 encodes, multiplexes, and/or digitizes the outgoing baseband data to generate a processed baseband or IF signal. The RF transceiver 310 receives the outgoing processed baseband or intermediate frequency signal from the TX processing circuitry 315 and up-converts the baseband or intermediate frequency signal to an RF signal that is transmitted via the antenna 305. In an embodiment, the two or more network access interfaces can include one or more I/O IFs 345, one or more RF transceivers 310, or the like. The I/O IF 345 can communicate via a wired connection such as a network interface card for an Ethernet connection or a cable interface for a set top box. The RF transceivers 310 can communicate with a wireless access point (such as wireless access point 118), a base station (such as base station 116), or the like.

The processor 340 can include one or more processors or other processing devices and execute the OS program 361 stored in the memory 360 in order to control the overall operation of the client device 300. For example, the processor 340 could control the reception of forward channel signals and the transmission of reverse channel signals by the RF transceiver 310, the RX processing circuitry 325, and the TX processing circuitry 315 in accordance with well-known principles. In some embodiments, the processor 340 includes at least one microprocessor or microcontroller.

The processor 340 is also capable of executing other processes and programs resident in the memory 360. The processor 340 can move data into or out of the memory 360 as required by an executing process. In some embodiments, the processor 340 is configured to execute the applications 362 based on the OS program 361 or in response to signals received from external devices or an operator. The processor 340 is also coupled to the I/O interface 345, which provides the client device 300 with the ability to connect to other devices such as laptop computers and handheld computers. The I/O interface 345 is the communication path between these accessories and the processor 340.

The processor 340 is also coupled to the keypad 350 and the display unit 355. The operator of the client device 300 can use the keypad 350 to enter data into the client device 300. The display 355 may be a liquid crystal display or other display capable of rendering text and/or at least limited graphics, such as from web sites.

The LED1 357 (at a given wavelength, λ₁) and the LEDs 358 (at an alternative wavelength, λ₂) are configured to emit light on a target region of a living body. A camera 359 is configured to capture an image of the target region while the LED1 357 and the LED2 358 emit light on the target region. The camera 359 can be a high resolution camera that is integrated with a thin pulsed light beam emitting LED sensors in a side-scatter configuration. The client device 300 can implement particle image velocimetry (PIV) and photoplethysmography (PPG) imaging systems to generate microvascular hemodynamic images of the target region to estimate blood pressure based on blood flow velocity, pulse oximetry, and heartrate variability.

The memory 360 is coupled to the processor 340. Part of the memory 360 could include a random access memory (RAM), and another part of the memory 360 could include a Flash memory or other read-only memory (ROM).

Although FIGS. 2 and 3 illustrate examples of devices in a communication system, various changes may be made to FIGS. 2 and 3. For example, various components in FIGS. 2 and 3 could be combined, further subdivided, or omitted and additional components could be added according to particular needs. As a particular example, the processor 340 could be divided into multiple processors, such as one or more central processing units (CPUs) and one or more graphics processing units (GPUs). Also, while FIG. 3 illustrates the client device 300 configured as a mobile telephone or smartphone, client devices could be configured to operate as other types of mobile or stationary devices. In addition, as with computing and communication networks, client devices and servers can come in a wide variety of configurations, and FIGS. 2 and 3 do not limit this disclosure to any particular client device or server.

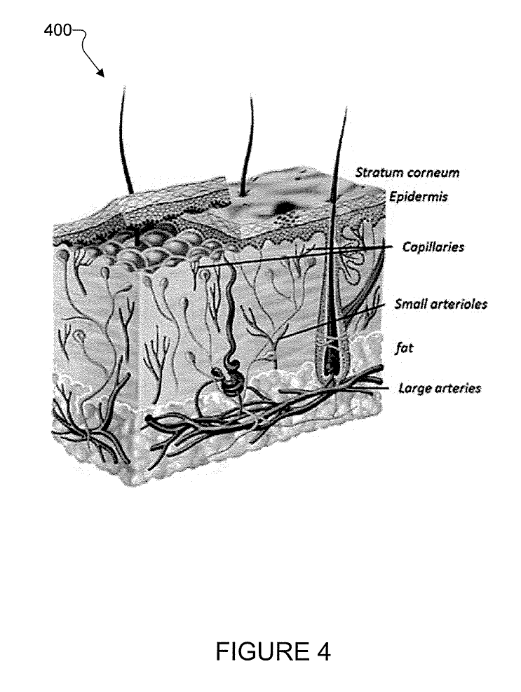

An electronic device can implement a combined microscopic PIV and PPG imaging system that share common components for imaging the narrow depth of field (DOF; 1-2 mm) zones of extremities for measuring blood flow velocity, pulse oximetry and heart rate variability. FIG. 4 illustrates an example cross-sectional diagram of anatomy of a human epidermal layer according to this disclosure. The diagram includes the location of blood vessels in the form of shallow capillaries, deep arterioles, and deeper large arteries. Ideal DOFs would contain capillaries and small blood vessels such as the palmar digital arteries in the hand. TABLE 1 provides physical properties of common arteries in the human hand and wrist.

TABLE 1
Physical properties of hand and wrist arterial vessels

| | Mean Diameter | Radius | Cross-sectional area | Length |
|---|---|---|---|---|
| Arteries | (cm) | (cm) | (cm²) | (cm) |
| Palmar digital | 0.085 | 0.0425 | 0.006 | 10 |
| artery | | | | |
| Radial | 0.254 | 0.127 | 0.051 | 18.1 |
| Ulnar | 0.212 | 0.106 | 0.035 | 18.5 |

Based on the physical parameters shown in Table 1, by using the Poiseuille-Hagen formula given in equation 1.1, mean arterial blood flow velocities are calculated on the basis of equation 1.2 and provided in TABLE 2.

$$Flow = {Δ}_{p} \frac{π}{8} \cdot \frac{1}{η} \cdot \frac{{r}^{4}}{L} \qquad 1.1$$

where Δp is the pressure difference or mean pressure (Pascals, Pa), η is the low shear rate viscosity (Poise, P), r is the radius, and L is the length of the vessel.

$${V}_{avg} = \frac{Flow}{A} \qquad 1.2$$

where V_{avg} is the mean velocity and A is the cross sectional area (cm²).

TABLE 2
Mean velocity of arterial flow in the hand and wrist

| | | Pressure | Pressure | Flow | Mean |
|---|---|---|---|---|---|
| | Viscosity | Difference | difference | (mL or | velocity |
| Arteries | (P) | (mm Hg) | (Pa) | cc/s) | (cm/s) |
| Palmar digital | 0.0524 | 20 | 2666 | 0.0665 | 1.1477 |
| artery | | | | | |
| Radial | 0.0524 | 80 | 10664 | 1.1481 | 22.6482 |
| Ulnar | 0.0524 | 80 | 10664 | 0.5451 | 15.4363 |

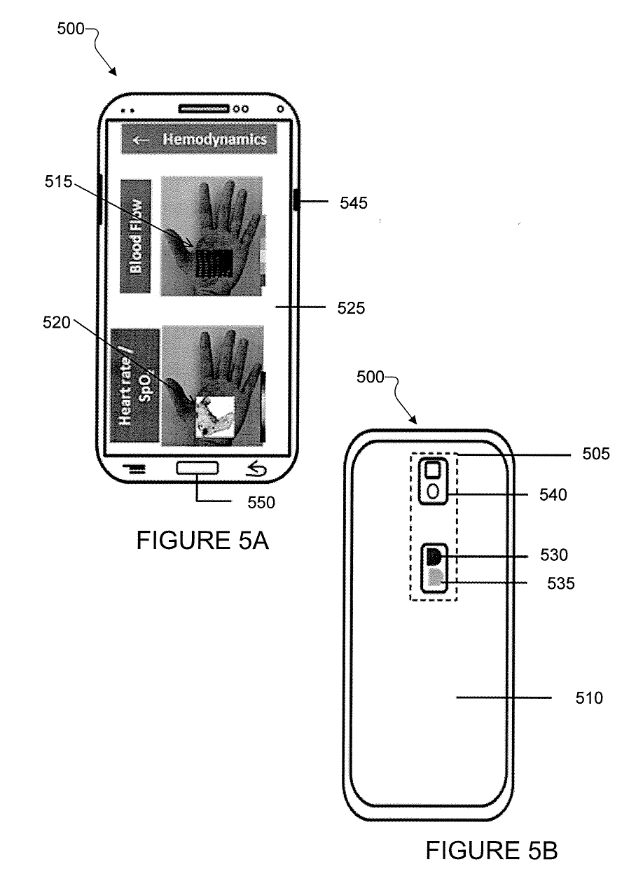

FIGS. 5A and 5B illustrate an example electronic device 500 including a combined PIV and PPG imaging system 505 according to this disclosure. FIG. 5A illustrates a front view of the example electronic device 500 and FIG. 5B illustrates a rear view of the example electronic device 500. As shown in FIGS. 5A and 5B, the electronic device 500 includes the combined PIV and PPG imaging systems 505 integrated into the rear case 510 of the electronic device 500 along with images 515 and 520 containing overlays of hemodynamic parameters such as blood flow, heart rate, and SpO₂ on a display 525. FIG. 5B illustrates two LEDs (a LED1 530 and an LED2 535) in line with an imaging camera 540. The electronic device 500 can also include a power button 545 and a home button 550.

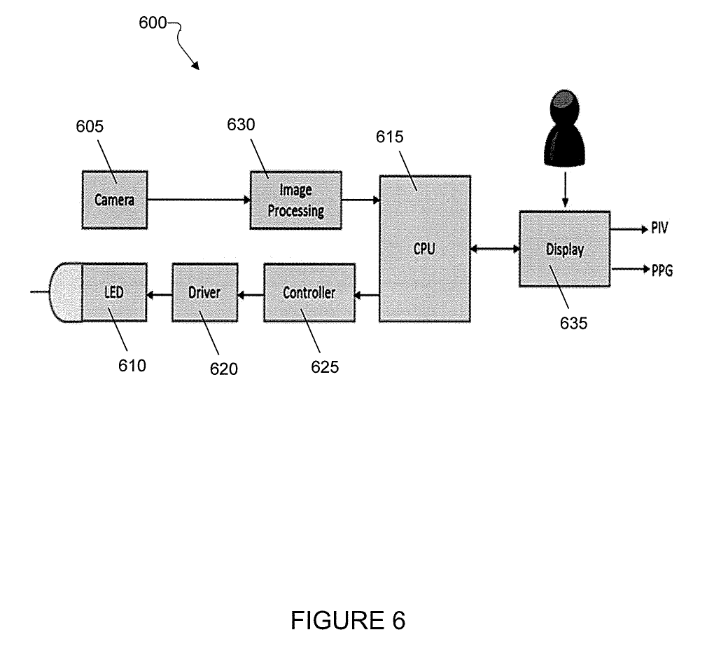

FIG. 6 illustrates an example system block diagram of an example electronic device 600 according to this disclosure. As shown in FIG. 6, a high-resolution camera 605 has been integrated with thin pulsed light beam emitting LED sensors 610 in a side-scatter configuration which minimizes the complexity and equipment overhead (as it is for backscatter and forward scatter designs) and also maximizes the unobtrusiveness of the system. Secondly, all image pre- and post-processing functions take place in the central processing unit (CPU) 615 compared to conventional PIV and PPG imaging systems where these tasks are preformed off-line and require a dedicated desktop or laptop computer. The electronic device 500 can also include a driver 620, a controller 625, an image processor 630, and a display 635. The electronic device 500 can be a smartphone or a tablet, for example.

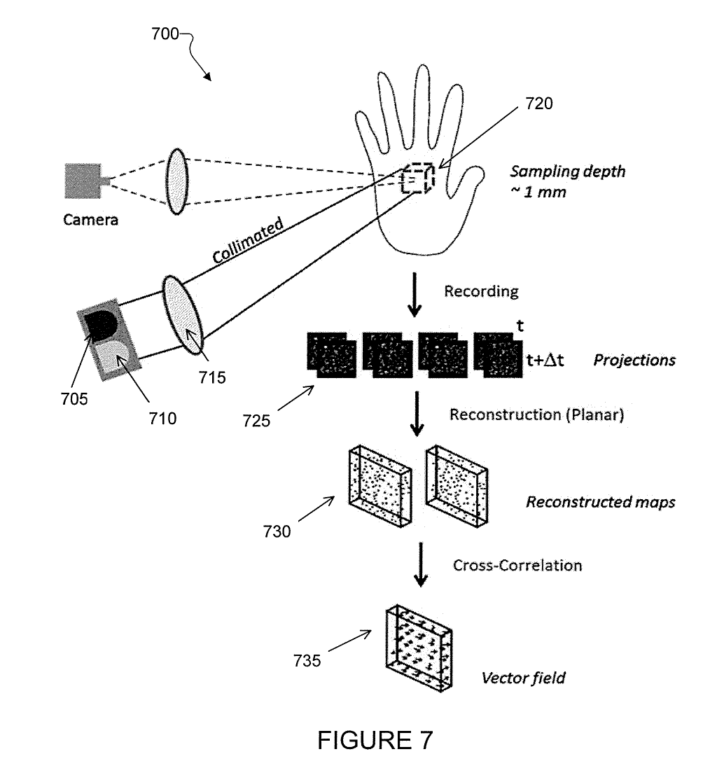

FIG. 7 illustrates an example microscopic PIV system 700 according to this disclosure. The microscopic PIV system 700 includes a side-scatter configuration for generating vector field maps of blood flow in anatomical regions such as the palm of the hand. The side-scatter configuration is used for implementing a microscopic PIV method in a smartphone or handheld device for example.

The system 700 includes at least two different high-power LEDs (an LED1 705 and an LED2 710) with the LED1 705 possessing a higher power output relative to LED2 710. The LEDs 705 and 710 are surface emitters with a nearly constant light distribution per unit area. They are used for volume illumination due to a large light emitting area. The LEDs 705 and 710 would be operated in a pulsed mode with the maximum current of ˜30 A. The LED1 705 and the LED2 710 emit light through a lens 715 that collimates the light rays onto the medium or sample area 720.

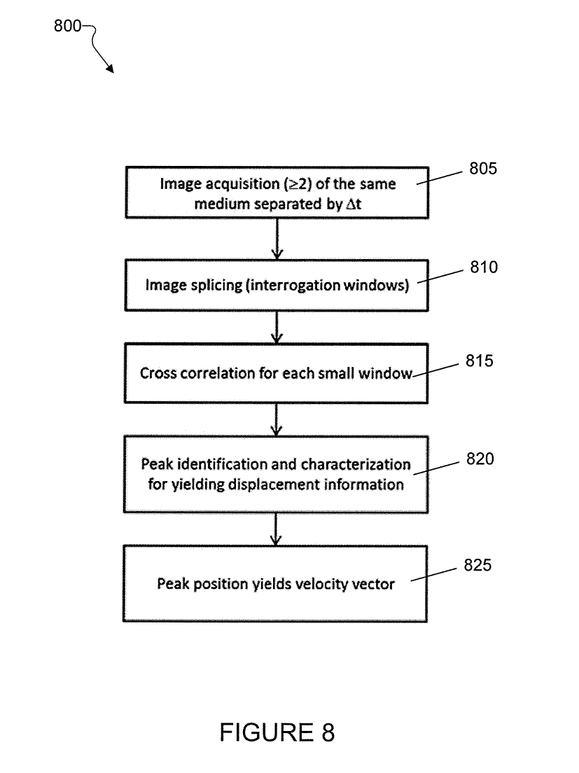

FIG. 8 illustrates an example method 800 implemented using a microscopic PIV system according to this disclosure. At step 805, two or more images 725 of the same medium 720 are acquired back-to-back and separated by a distinct time interval (Δt). At step 810, these images 720 are spliced into small regions referred to as interrogation windows 730. At step 815, a cross-correlation between two successive images 720 is calculated for each small window 730. At step 820, peak identification and characterization are then performed in the cross-correlation image 735. At step 820, a peak location yields the displacement for which the two images are most similar, such as the amount by which the second image has to be moved in order to appear as the first image (prior to the occurrence of any flow). The velocity vector is defined as the peak's position. This follows the notion that the image between two successive time intervals did not change drastically in content but was moved or deformed.

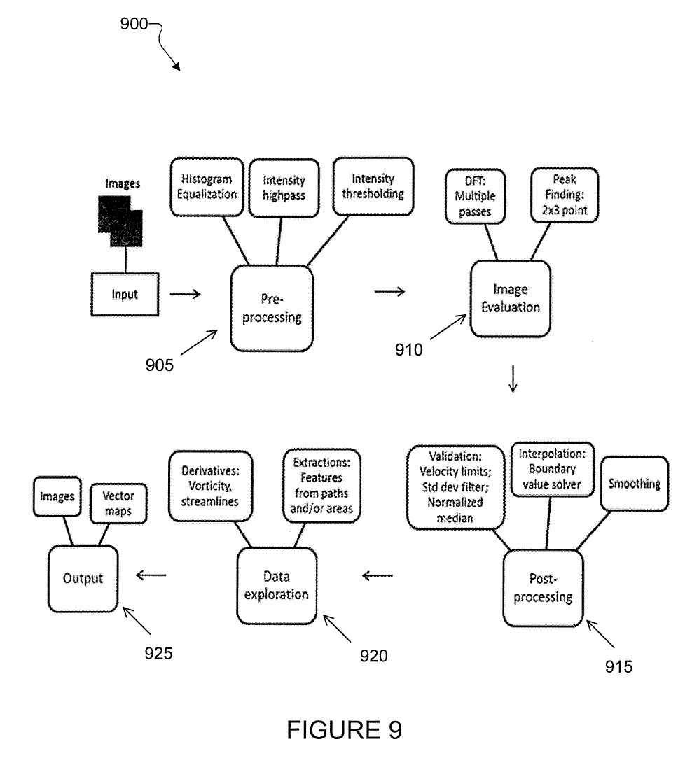

FIG. 9 illustrates an example method 900 of image sensing using a microscopic NV system according to this disclosure. At step 905, PIV analysis can be condensed into image pre-processing, image evaluation, post-processing, data extrapolation, and output. The workflow initiates from the left with image input and pre-processing functions and then continues on to the right with evaluation at step 910, post-processing at step 915, data extrapolation at step 920, and output at step 925. A core function of the pre-processing task is image enhancement to improve the measurement quality of the data prior to image correlations. Histogram equalization is undertaken to optimize image regions with low exposure and high exposure independently by spreading out the most frequent intensities of the image histogram to the full range of the data (0-255 in 8-bit images). A highpass filter is applied to address inhomogeneous lighting for keeping particle information in the image and suppressing low frequency information. Pre-processing entails image thresholding to address statistical biases in the images due to the presence of bright particles within an area which can confound the correlation signal. For this reason, an upper limit of the grayscale intensity is chosen and pixels that exceed this threshold are replaced by the upper limit. These three sub-processes of the image pre-processing step improve the probability of detecting valid vectors.

The next task comprises image evaluation of which the cross-correlation algorithm is the most sensitive part. Small sub-images or interrogation areas of an image pair are cross correlated to derive the most probable particle displacement in these areas. A correlation matrix can be computed in the frequency domain by means of the discrete Fourier transform (DFT) calculated using a fast Fourier transform (FFT). The interrogation grid can be refined with each pass providing a high spatial resolution in the final vector map along with a high dynamic velocity range and signal-to-noise ratio. The first pass provides displacement information in the center of an interrogation area. When the areas overlap one another by 50% or so, there is additional displacement information at the borders and corners of each interrogation area. Bilinear interpolation allows calculation of displacement information at every pixel of the interrogation regions. The next interrogation area is deformed according to this displacement information. Subsequent interrogation passes correlate the original interrogation area with the newly deformed area. Between passes, the velocity information is smoothed and validated. For peak finding, the integer displacement of two interrogation areas can be determined straightforward from the location of the intensity peak of the correlation matrix. The process involves fitting a Gaussian function to the integer intensity distribution. The peak of the fitted function enables determination of the particle displacement with sub-pixel accuracy.

The next task encompasses post-processing where outliers are filtered based on velocity thresholds. These thresholds can be set arbitrarily or can be based on a local median filter implementation where the velocity fluctuations are evaluated in a 3×3 neighborhood around a central vector with the median of such fluctuations used as normalization for a more classical median test. After this step, missing vectors can be replaced by interpolated data, e.g. by a 3×3 neighborhood interpolation. To address the reduction of measurement noise, data smoothing can be applied by means of median filtering. The final output can take the form of vectograms or vector field maps showing complex flow patterns or quantitative images depicting derivatives such as vorticity and divergence from paths or areas.

The microscopic PIV system parameters are given in TABLE 3. The interrogation window size depends on the density of the particle images. In a cross-correlation of a pair of two single exposed recordings, Xᵢ can be considered to be the position vector and xᵢ can be considered as the image position vector of a particle i (such as a red blood cell) in the first exposure. They are related as:

$${X}_{i} = \frac{{x}_{i}}{M} \qquad 1.3$$

where M is the magnification factor. The image intensity field of the first exposure can be expressed as:

$$I ( x ) = {\sum }_{i = 1}^{N} {V}_{0} ( {X}_{i} ) τ ( x - {x}_{i} ) \qquad 1.4$$

where V₀(Xᵢ) is the transfer function yielding the light energy of the image of an individual particle, I, inside the interrogation volume and its conversion into an electric signal. τ(x) is the point spread function of the imaging lens assumed to be Gaussian in both directions of the plane.

If we assume that between two interrogation windows, all particles have moved with the same displacement vector, ΔX, the image intensity field of the second exposure may be expressed as:

$${I}^{′} ( x ) = {\sum }_{j = 1}^{N} {V}_{0}^{′} ( {X}_{j} + Δ X ) τ ( x - {x}_{j} - δ x ) \qquad 1.5$$

where δx is the particle image displacement which could be approximated by:

$$Δ X = \frac{δ x}{M} \qquad 1.6$$

The cross-correlation of the two interrogation windows can be defined as:

R(s)=<I(x)I(x+s)> 1.7

where s is the separation vector in the correlation plane and < > is the spatial averaging operator over the interrogation window. R can be decomposed into three components as:

R(s)=R_{c}(s)+R_{F}(s)+R_{D}(s) 1.8

where R_{c} is the correlation of the mean image intensities and R_{F} is the noise component (due to fluctuations), both resulting from i≠j terms. The displacement cross-correlation peak, R_{D}, represents the component of the cross-correlation function that corresponds to the correlation of images of particles from the first exposure with images of identical particles present in the second exposure (i=j terms). The peak reaches a maximum for s=δx. The determination of this location of the maximum yields δx, thus ΔX. This location is usually obtained by systematic exploration of the interrogation windows on the basis of FFT algorithms for cross-correlations.

TABLE 3
List of microscopic PIV system parameters

| | Pulsed, high- | | |
|---|---|---|---|
| Flow | power LED | Camera | Image properties |
| Mesh | Pulse width, | Resolution, | Lens focal length, 28 mm |
| size, | 150 μs | 5312 × 2988 | Viewing angles, 30°, ±45° |
| 10 mm | Max pulse | pixels | Aperture, 12 |
| | current, | Video, 1080p | Diffraction limit, 4 μm |
| | 30 A ~Pulse | @ 60 fps | Image magnification, 15x |
| | energy, 2.0-5.0 | 2.0 Megapixels | Particle image diameter, |
| | mJ Pulse | (1920 × 1080) | 8 μm |
| | separation, | Acquisition | Field of view (FOV), 25 × |
| | 5 ms | rate, 1 Hz | 25 mm² |
| | | | Max. particle displacement, |
| | | | 20 pixels |

The flow velocity derived from the microscopic PIV system can be used to estimate the pulse wave velocity (PWV), defined as the speed of propagation of a blood pressure pulse. PWV, which is proportional to arterial stiffness, is typically determined from the combination of an electrocardiogram R-wave and a blood pressure cuff or a PPG sensor in the form of an LED and photodetector. However, the Water Hammer equation can also yield an alternate expression of PWV. This equation related PWV through the ratio of pressure (Δp) and linear velocity (v) in the absence of wave reflection.

$$PWV = \frac{Δ p}{v ρ} \qquad 1.9$$

where ρ is the density of blood. The traditional form of PWV is given on the basis of the Moens-Kortewegg equation as:

$$PWV = \sqrt{\frac{gtE}{ρ d}} \qquad 1.10$$

where E is the elasticity of the vessel wall which can be treated as the elastic modulus at zero pressure, t is the arterial thickness, d is the arterial diameter and g is the gravitational constant. The pulse transit time (PTT), the time taken for a pulse wave to travel between two arterial sites, is related to PWV in the form of:

$$PWV = \frac{K}{PTT} \qquad 1.11$$

where K is a proportional coefficient indicating the distance that the pulse has to travel between two arterial locations. An alternative embodiment for characterizing the PWV could be on the basis of using the two PPG sensors (LEDs and associated photodiodes) without employing micro PIV. For such a measurement to be effective, both sensors would need to be abutted parallel to a superficial artery such as the palmar digital artery. The pulse transit distance, K, between the two sensors is then measured as the distance between the up-stream edges of the two photodiodes. For the current hardware configuration, K would vary between about 5-10 cm, with the sampling rate being inversely proportional to K. PTT of the pressure pulse is then measured as the difference in time between the time of the onset of the pulse wave observed at the distal sensor (such as a sensor that is closer to the extremities) and the time of the onset of the pulse at the proximal sensor (such as a sensor that is closer to a wrist), given by equation 1.11. The end point blood pressure (Pₑ) can be related to PTT directly by:

$${P}_{e} = {P}_{b} - \frac{2}{γ {PTT}_{b}} Δ PTT \qquad 1.12$$

where P_{b} is the base blood pressure level, PTT_{b} is the value of PTT corresponding to that pressure (P_{b}) and ΔPTT is the change in the PTT.

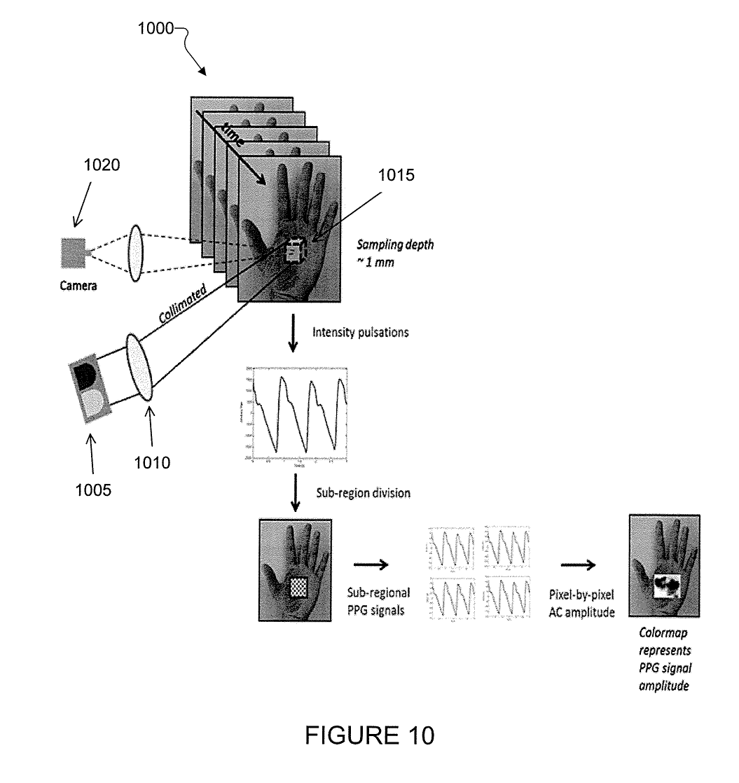

A combined PPG imaging system can be used that utilizes the same electronic device as the micro-PIV system (as shown in FIG. 7). FIG. 10 illustrates an example of a PPG imaging system 1000 according to this disclosure. Pulsed LEDs 1005 are used in concert with a lens 1010 to generate collimated thin light beams illuminating the desired anatomical region 1015 (such as a palm). A high-frame rate camera 1020 then captures volumetric changes in blood flow of superficial blood vessels at a certain distance (˜10 cm) from the region 1015 over a small field of view (25×25 mm²) at a sampling depth of ˜1 mm. This allows recording changes in transmitted or reflected light which allow measuring intensity pulsations from heart beat to heart beat. Further, image processing tasks encompassing sub-regional analyses as discussed herein permit estimation of the pixel-by-pixel variations in the PPG signal amplitude. Additionally, the oxygen saturation can also be computed on a pixel-by-pixel basis by calculating the ratio of (λ1) and (λ2) LED lights absorbed by the blood.

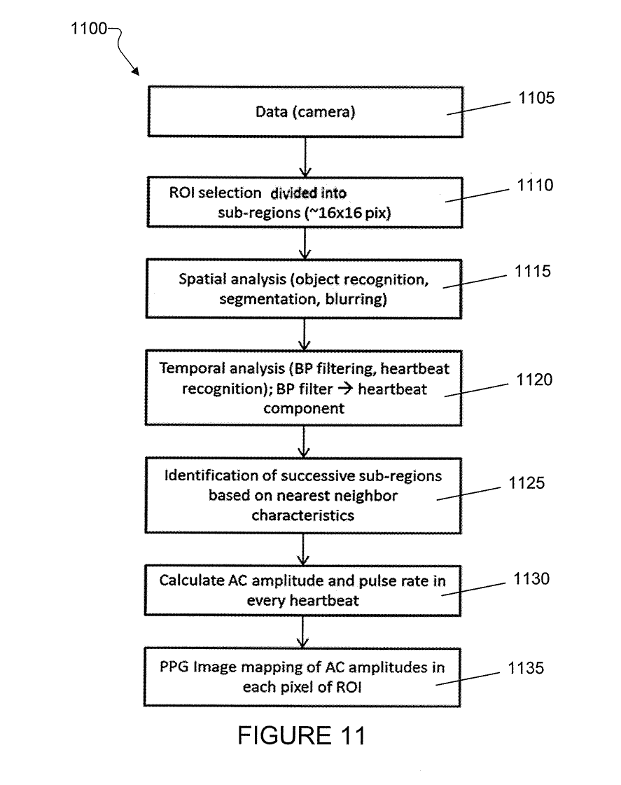

FIG. 11 illustrates an example method 1100 to compute a final PPG imaging color maps for displaying the AC amplitude of the PPG signal on a pixel-by-pixel basis according to this disclosure. At step 1105, recorded data from the camera is filtered and pre-processed. At step 1110, a region of interest (ROI) on the anatomy of interest is selected and subdivided into an array of pixels. At step 1115, this ROI then undergoes spatial analysis encompassing object recognition, segmentation and blurring. At step 1120, temporal analysis is undertaken consisting of blood pressure filtering and heartbeat recognition for identifying beat-to-beat components. At step 1125, the identification of successive sub-regions is undertaken based on nearest neighbor characteristics. At step 1130, a computation of the AC amplitude and pulse rate in every heartbeat is performed. At step 1135, the final output consists of a color map yielding the amplitude of the PPG signal in each pixel of an ROI.

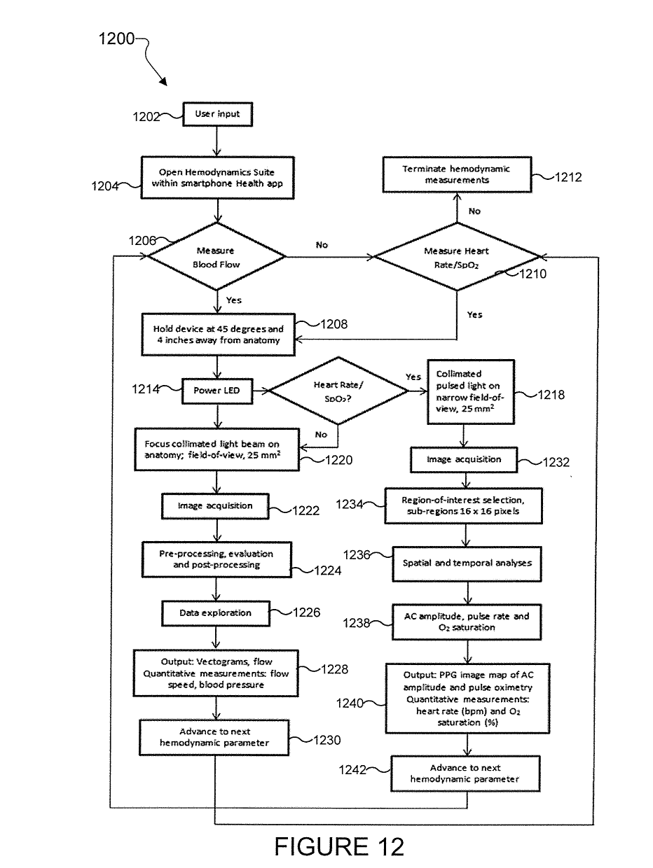

FIG. 12 illustrates an example method 1200 for demonstrating the operation of the image sensors when combining the PIV and PPG imaging systems using an electronic device according to this disclosure. At step 1202, a user input is provided to an electronic device. At step 1204, a hemodynamic suite is open on the electronic device application. At step 1206, the application outputs a request asking whether a user wants to measure blood flow. At step 1210, if an input is provided not requesting to measure blood flow, the output is provided asking whether a user want to measure heartrate and blood oxygen concentration. At step 1212, if a input is provided not requesting to measure heartrate and blood oxygen concentration, then hemodynamic measurements are terminated. At step 1208, if an input is provided requesting to measure blood flow or to measure heartrate and blood oxygen concentration, the electronic device produces an output directing a user to hold the electronic device at a 45 degree angle and 4 inches away from body target area.

At step 1214, an LED on the electronic device is powered on. At step 1218, if heartrate and blood oxygen concentration is being measured, then the electronic device outputs an indication to collimate pulsed light on a narrow field-of-view (such as 25 mm²). At step 1232, an image is acquired. At step 1234, a region of interest is selected and divided into sub regions of, for example, 16×16 pixels. At step 1236, spatial and temporal analysis is performed by the electronic device. At step 1238, the electronic device calculates AC amplitude, pulse rate, and oxygen saturation or concentration. At step 1240, the electronic device outputs a PPG image map of AC amplitude and pulse oximetry quantitative measures includes heart rate and oxygen concentration or saturation. At step 1242, if blood flow is being measured, then the electronic device provides an indication to focus collimated light beams on a narrow field-of-view (such as 22 mm²). At step 1222, the electronic device acquires images. At step 1224, the electronic device performs pre-processing, evaluation, and post-processing. At step 1226, the electronic device performs data exploration. At step 1228, the electronic device outputs vectograms and flow quantitative measurements includes flow velocity and blood pressure. At step 1230, the electronic device can advance to measure another hemodynamic parameter.

These modalities would be integrated into existing health applications suites and be utilized as part of an ensemble of sensors that are available for monitoring various physiological parameters. The image acquisition and processing elements for each modality in this combined setup would utilize the workflow shown in FIGS. 7, 8, and 9 for PIV and FIGS. 10 and 11 for PPG imaging.

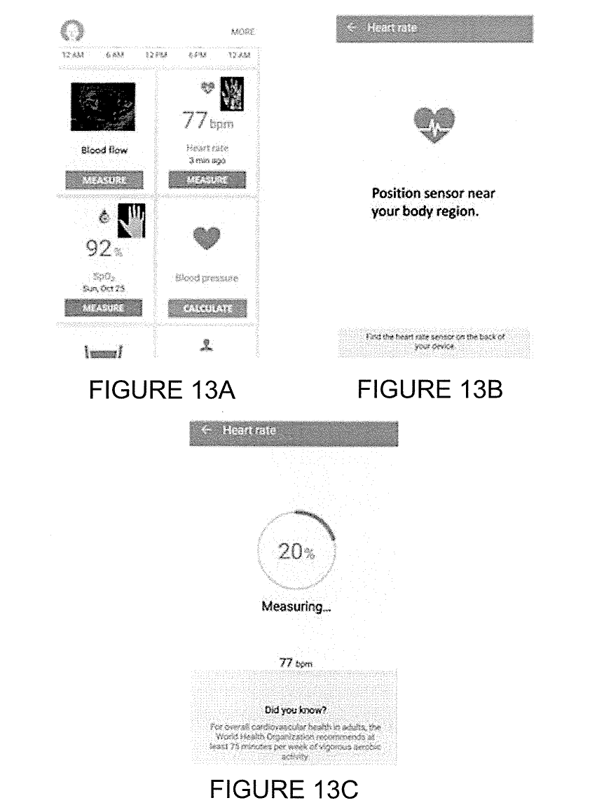

FIGS. 13A, 13B, and 13C illustrate an example visualization depicting a user interface on an electronic device according to this disclosure. FIG. 13A illustrates a user interface for measuring blood flow (such as by micro PIV), heart rate and SpO₂ (such as by PPG imaging), and ultimately for calculating blood pressure. FIG. 13B illustrates an example panel that shows instructions given for positioning sensors. FIG. 13C illustrates an example panel that shows an indication that heart rate measurements are underway.

Given that the combination of these two systems, PIV and PPG imaging in a mobile device would provide several hemodynamic parameters such as blood perfusion status, flow speed, blood pressure (extrapolated from velocity, pulse wave velocity and pulse transit time), heart rate and oxygen saturation, and the like, this device can now be treated as a ‘cuff-less’ blood pressure monitoring system in healthy individuals and those afflicted with cardiovascular diseases such as heart attacks, congestive heart failure, coronary artery disease, and/or individuals with pacemakers and those discharged and needing to be monitored following heart surgery. Healthy individuals who are interested in self-monitoring and quantification of their biometrics would be able to track their hemodynamic parameters on a longitudinal basis for tracking their health or sharing with their medical providers. The device would also be a gateway for healthcare professionals to monitor vital hemodynamic parameters remotely in ambulatory patients who need to be monitored for several days and weeks following discharge from a clinic or hospital.

Furthermore, given the device's ability to characterize heart rate and heart rate variability (HRV), an electronic device can also serve as a continuous HRV monitor in individuals who need to monitor their HRV status closely due to stress, fatigue, and insomnia which also tend to affect healthy individuals from time to time. Additionally because of the abundance of hemodynamic parameters generated by the PIV and PPG systems, the electronic device serves as a blood circulation monitor in individuals being monitored closely for formation of blood clots that can cause heart attacks or stroke by traveling to the brain. Here, parameters such as blood flow speed, blood pressure, vessel wall tension and capacitance will factor in for successful monitoring of such patient populations. Lastly, the combined PIV and PPG systems also offer the potential to monitor disorders such as Raynaud's syndrome where individuals suffer from excessively poor blood flow in their hands, fingers, toes and other areas due to cold temperatures or emotional stress. Here, parameters such as flow speed, blood pressure, PPG imaging maps and oxygen saturation maps would provide visual and quantitative feedback to the users to then relay the information to their healthcare providers.

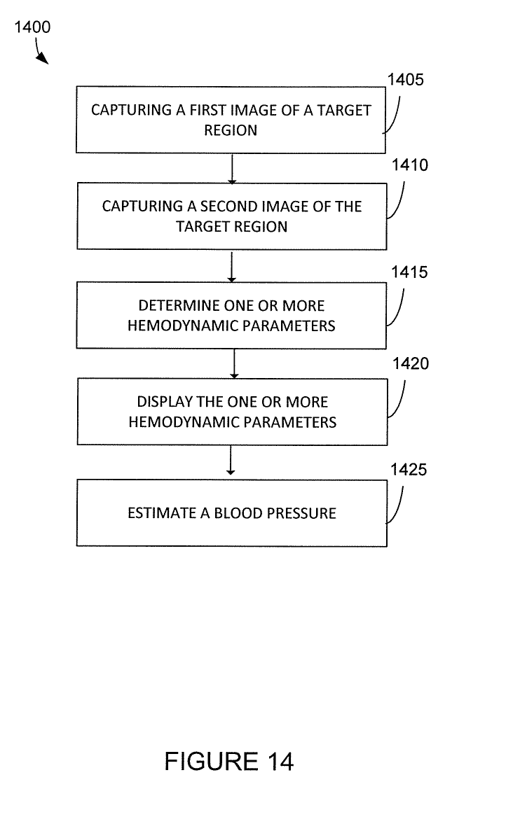

FIG. 14 illustrates an example method 1400 to measure microvascular hemodynamic parameters according to this disclosure. At step 1405, a device captures, using a camera, a first image of a target region while a pair of light emitting diodes (LEDs) emit light on the target region. The camera can be a high resolution camera. At step 1410, the device captures, using the camera, a second image of the target region while the LEDs emit light on the target region. The second image is captured a predetermined time after the first image is captured. At step 1415, the device determines one or more hemodynamic parameters based on a difference between the first captured image the second captured image. At step 1420, the device displays, on a display, the one or more hemodynamic parameters over a displayed image of the target region. At step 1425, the device estimates blood pressure based on the one or more hemodynamic parameters.

Although the present disclosure has been described with an exemplary embodiment, various changes and modifications may be suggested to one skilled in the art. It is intended that the present disclosure encompass such changes and modifications as fall within the scope of the appended claims.

What is claimed is:

1. A device to measure hemodynamic parameters, the device comprising:
a first light emitting diode (LED) sensor configured to emit light at a first wavelength (λ₁);
a second LED sensor configured to emit light at a second wavelength (λ₂), wherein the first LED sensor and the second LED sensor are covered with a collimated lens;
a camera; and
a processor configured to:
control the camera to capture a first image of a plurality of images of a target region while the first LED sensor and the second LED sensor emit light on the target region;
control the camera to capture a second image of the plurality of images of the target region while the first LED sensor and the second LED sensor emit light on the target region, wherein the second image is captured a predetermined time after the first image is captured; and
determine one or more hemodynamic parameters based on a difference between at least the first captured image and the second captured image of the plurality of images.

2. The device of claim 1, wherein the first LED sensor and the second LED sensor are thin pulsed light beam emitting LED sensors, and wherein the camera is integrated with the first LED sensor and the second LED sensor in a side-scatter configuration.

3. The device of claim 1, further comprising a display configured to display the one or more hemodynamic parameters over a displayed image of the target region.

4. The device of claim 1, wherein the one or more hemodynamic parameters comprises at least one of a magnitude and direction of blood flow, a heartrate, or an oxygen saturation level.

5. The device claim 1, wherein the device comprises at least one of a smartphone or a tablet.

6. The device of claim 1, wherein the processor is further configured to estimate a blood pressure based on the one or more hemodynamic parameters.

7. The device of claim 1, wherein the processor is configured to determine the one or more hemodynamic parameters based on the difference between at least the first captured image the second captured image of the plurality of images by:
splicing each of at least the first image and the second image of the plurality of images into a plurality of image regions;
cross-correlating each of the plurality of regions between at least the first image and the second image of the plurality of images;
identifying peaks based on the cross-correlation of the plurality of regions between at least the first image and the second image of the plurality of images; and
identifying one or more velocity vectors within the target region for particle image velocimetry (PIV) image.

8. The device of claim 1, wherein the processor is configured to determine the one or more hemodynamic parameters based on the difference between at least the first captured image the second captured image of the plurality of images by:
splicing each of at least the first image and the second image of the plurality of images into a plurality of image regions;
performing a spatial analysis on each of the plurality of image regions for at least the first image and the second image of the plurality of images;
performing a temporal analysis on each of the plurality of image regions for at least the first image and the second image of the plurality of images, wherein the temporal analysis includes at least one of blood pressure filtering or heartbeat recognition; and
generating data for a color map for a photoplethysmography (PPG) image.

9. A device to measure hemodynamic parameters, the device comprising:
a first light emitting diode (LED) sensor configured to emit light at a first wavelength (λ₁);
a second LED sensor configured to emit light at a second wavelength (λ₂), wherein the first LED sensor and the second LED sensor are covered with a collimated lens;
a camera; and
a processor configured to:
control the camera to capture a first image of a plurality of images of a target region while the first LED sensor and the second LED sensor emit light on the target region;
control the camera to capture a second image of the plurality of images of the target region while the first LED sensor and the second LED sensor emit light on the target region, wherein the second image is captured a predetermined time after the first image is captured;
receive a selection to perform at least one of particle image velocimetry (PIV) imaging or photoplethysmography (PPG) imaging; and
determine one or more hemodynamic parameters based on (1) a difference between at least the first captured image and the second captured image of the plurality of images and (2) the received selection.

10. The device of claim 9, wherein the first LED sensor and the second LED sensor are thin pulsed light beam emitting LED sensors, and wherein the camera is integrated with the first LED sensor and the second LED sensor in a side-scatter configuration.

11. The device of claim 9, further comprising a display configured to display the one or more hemodynamic parameters over a displayed image of the target region.

12. The device of claim 9, wherein the one or more hemodynamic parameters comprises at least one of a magnitude and direction of blood flow, a heartrate, or an oxygen saturation level.

13. The device of claim 9, wherein the device comprises at least one of a smartphone or a tablet.

14. The device of claim 9, wherein the processor is further configured to estimate a blood pressure based on the one or more hemodynamic parameters.

15. The device of claim 9, wherein the processor is configured to, after receiving a selection to perform particle image velocimetry (PIV) imaging, determine the one or more hemodynamic parameters based on the difference between at least the first captured image the second captured image of the plurality of images by:
splicing each of at least the first image and the second image of the plurality of images into a plurality of image regions;
cross-correlating each of the plurality of regions between at least the first image and the second image of the plurality of images;
identifying peaks based on the cross-correlation of the plurality of regions between at least the first image and the second image of the plurality of images; and
identifying one or more velocity vectors within the target region for particle image velocimetry (PIV) image.

16. The device of claim 9, wherein the processor is configured to, after receiving a selection to perform photoplethysmography (PPG) imaging, determine the one or more hemodynamic parameters based on the difference between at least the first captured image the second captured image of the plurality of images by:
splicing each of at least the first image and the second image of the plurality of images into a plurality of image regions;
performing a spatial analysis on each of the plurality of image regions for at least the first image and the second image of the plurality of images;
performing a temporal analysis on each of the plurality of image regions for the first image and the second image of the plurality of images, wherein the temporal analysis includes at least one of blood pressure filtering or heartbeat recognition; and
generating data for a color map for a photoplethysmography (PPG) image.

17. A method implemented by a device to measure hemodynamic parameters, the method comprising:
capturing, by a camera, a first image of a plurality of images of a target region while two light emitting diode (LED) sensors differing in wavelength emit light, via a collimated lens, on the target region;
capturing, by the camera, a second image of the plurality of images of the target region while the two LED sensor emit light, via the collimated lens, on the target region, wherein the second image is captured a predetermined time after the first image is captured; and
determining one or more hemodynamic parameters based on a difference between at least the first captured image and the second captured image of the plurality of images.

18. The method of claim 17, further comprising displaying the one or more hemodynamic parameters over a displayed image of the target region.

19. The method of claim 17, wherein the one or more hemodynamic parameters comprises at least one of a magnitude and direction of blood flow, a heartrate, or an oxygen saturation level.

20. The method of claim 17, further comprising estimating blood pressure based on the one or more hemodynamic parameters.
